(19) United States
(12) Patent Application Publication
(10) Pub. No.: US 2012/0296342 A1
(43) Pub. Date: Nov. 22, 2012

(54) ELECTRIC HAND-HELD GROOMING TOOL

(76) Inventor: Kathleen Haglund Wendelschafer, San Tan Valley, AZ (US)

(21) Appl. No.: 13/327,362

(22) Filed: Dec. 15, 2011

Related U.S. Application Data
(60) Provisional application No. 61/488,760, filed on May 22, 2011.

(51) Int. Cl. A61B 17/50

(52) U.S. Cl. 606/131

(57) ABSTRACT

An electric hand-held grooming tool is provided, which has a body portion, including a neck portion and an attachment portion, and attachments that are structured to releasably couple to the attachment portion of the body portion. The body portion houses a motor that operates to provide rotary motion to the attachments. The body portion further includes an on/off control for actuating the device and a plurality of speed controls for changing the rotational speed of the motor and thus the attachments coupled thereto. When actuated, the motor of the device rotates the various attachments about their respective axis of rotation such that a user can place the rotating attachments against her skin to treat dry or callused skin surfaces. The motor may also be configured to provide a vibrating motion in addition to the rotational motion.

### BACKGROUND OF THE INVENTION

1. Technical Field

This invention relates generally to grooming products. More particularly, this invention relates to a device and method for removing dead skin from areas of the body.

2. State of the Art

In today's world, people are constantly on the move. Whether at home, at work, or at play, we, on average, take a staggering eight to ten thousand steps per day. Unfortunately for our feet, most of our steps are taken in impossibly-tall high heels, oxfords with little or no cushioning, or shoes that may or may not fit properly. Needless to say, our feet are typically put through endless abuse. No wonder we complain of our feet being tired, achy, or sore. However, our feet aren't the only areas of our bodies that take constant wear and tear. Our hands, elbows, and knees, for example, take routine abuse on a daily basis as we go about our everyday tasks.

Luckily, we can treat our skin to a little tender-loving-care. Lotions and creams applied routinely to the skin can soothe, moisturize, and possibly keep our skin from becoming dry and cracked, and, in some instances, can even prevent calluses from forming. But, perhaps the most beneficial treatment to our skin, and in particular to our feet, is a pedicure.

Although most of us consider a pedicure as a luxurious indulgence, pedicures should be viewed as a healthy part of routine skin care. Most pedicures involve soaking the feet in warm water, massaging the feet, removing the dead skin around the foot, and trimming the toenails. Removing dead skin, in the form of calluses and dry patches, from the foot keeps the foot healthy and attractive. Removing dead skin is conventionally done through the use of a pumice stone or sponge, or other lightweight, fibrous object. Salon professionals, health-care providers, and household consumers alike utilize pumice stones as a safe and effective means to care for the skin by removing dry, flaky skin, as well as the thickened skin of calluses and corns.

Although effective in removing dry and callused skin, using a pumice stone to care for skin can have its drawbacks. The effective use of a pumice stone requires rapidly and repeatedly swiping the pumice stone or sponge back and forth across the designated area of the body. Swiping the pumice stone or sponge back and forth can be a slow and exhausting process, especially when removing large calluses. Indeed, the entire process can amount to extensive manual labor, resulting oftentimes in unwanted side effects of sore hands and wrists. These side effects often prevent users from caring for their own skin, it being hardly worth the effort.

Thus, to avoid the intense labor required to care for dry skin using a pumice stone, many consumers instead choose to dedicate a substantial portion of a day to having a salon, or other skin-care professional, perform the required work. However, having a salon perform the requisite skin care can be an expensive endeavor, and repeat visits to the salon add to the expense. In addition to the cost, dedicating several hours to a salon can be an inconvenience to a consumer's time that is otherwise obligated by life's responsibilities, such as caring for a family. Moreover, in the case of some working professionals, dedicating several hours to a salon is simply not an option.

Professional skin-care providers, on the other hand, cannot escape the extensive manual labor associated with their line of work, i.e., providing quality skin-care services. In fact, for the professional skin-care provider, the extensive manual labor associated with providing high-quality skin care is amplified by the repetitive work of caring for the dry skin of customer after customer.

Therefore, there is a need in the skin-care market for a device and method that offers professional skin-care providers a reprieve from the extensive manual labored required to care for their customers' dry skin. There is also a need in the skin-care market for a device and method that provides the average consumer a relatively inexpensive, effective, and convenient alternative to both the manual labor required for at-home skin care and the extra cost and time of the professional salon. Indeed, there is a need for an efficient, effective, easy-to-use skin care product that enables professional skin-care providers, health-care providers, and consumers alike to easily, efficiently, and properly care for skin by removing dry skin and calluses from feet, elbows, hands, and knees.

### SUMMARY OF THE INVENTION

The present invention relates to grooming products. More particularly, the invention relates to a device and method for removing calluses and dead skin from areas of the body.

The present invention provides an electronic hand-held skin care device comprising a body portion, including a neck portion and an attachment portion, and attachments that are structured to releasably couple to the attachment portion of the body portion. The body portion may further comprise a motor housed within the body portion that operates to provide rotary motion to the attachments. The body portion may further comprise an on/off control for actuating the device and a plurality of speed controls for changing the rotational speed of the motor and thus the attachments coupled thereto. When actuated, the motor of the device rotates the various attachments about their respective axis of rotation such that a user can place the rotating attachments against her skin to treat dry skin surfaces. The motor may also be configured to provide a vibrating motion in addition to the rotational motion.

One aspect of the present invention may include the plurality of speed controls that allow the user to change the operational speed of the attachments during use. That is, the user can treat her dry skin surfaces at an initial speed and then switch to different speeds by operating one of the plurality of speed controls, as desired, to continue treatment of the surfaces at a different speed, all while the device continues to operate. Moreover, the body portion of the device may have a contoured shape to fit comfortably into a user's hand to allow the user to conveniently reach and operate each of the plurality of speed controls.

Another aspect of the present invention may include a combined on/off and speed adjustment control that allows the user to control the actuation and rotational speed of the device with one control. Having one combined control simplifies the operation of the device and provides the user added benefits, such as allowing the user to actuate the device and adjust the rotational speed with one motion and without having to locate and operate additional controls of the device. Moreover, the combined control is configured to dynamically adjust the rotational speed of the motor and thus the attachments. That is, the combined control may be a sliding control that slides back and forth (up and down) to dynamically adjust the rotational speed according to the movement of the combined control. Such a configuration allows the user to finely adjust the rotational speed of the attachment connected thereto between a high-end speed and a low-end speed. Different attachments may require different speeds to operate according to the desires of the user.

Another aspect of the present invention may include a battery chamber within the body portion, the battery chamber being structured to hold one or more batteries that may provide power to the device. The battery chamber may be accessible to the user so that the user can replace the batteries as needed. On the other hand, the battery chamber may house rechargeable batteries that the user does not need to replace, but simply needs to recharge. In this regard, the present invention may further comprise a detachable power cord that may operate on, for example, conventional 110 V, 60 Hz (in the USA) power. The power cord, when properly connected to the device and a power outlet, operates to charge the rechargeable battery while providing power to the device so the user can operate the device while the rechargeable battery charges. The user may also connect the power cord to the device without operating the device to allow the rechargeable battery to charge.

Another aspect of the present invention may include a digital display that displays to the user the operational status of the device. The digital display may remain inactive until the device is operated. The digital display may also illuminate under the condition that the user operates any of the controls on the device, whether or not the device is currently powered on. The digital display is capable of concurrently displaying any of for example, the rotational speed of the attachment, the percentage of charge remaining in the battery, or the time left to operate the device until the charge in the battery is depleted.

Another aspect of the present invention may include the body portion being waterproof, or at least water-resistant, such that the user can operate the device in wet environments, such as, for example, the shower or bath. Moreover, the waterproof nature of the body portion further contemplates that the battery chamber, the digital display, the mating of the attachments to the body portion, and the plurality of controls, including the speed controls, the on/off control, and the combined control, are also structured to be waterproof or water-resistant such that the user can be confident in operating the device in wet environments.

Another aspect of the present invention may include each of the attachments further comprising a shaft, one end of the shaft being integral to the attachments and an other end of the shaft being structured to couple to the attachment portion of the device, so that under the condition that the shaft is coupled to the device the shaft rotates with the rotational input of the motor to rotate the attachment.

Another aspect of the present invention may include the attachments having the shaft being integral to the attachment portion of the device and the heads of the attachments being releasably and repeatedly coupled to the shaft, such that under the condition that the attachment head is coupled to the shaft the attachment rotates with the rotational input of the motor.

Another aspect of the present invention may include the attachments being made of pumice stone or other hard abrasive material, such as aluminum oxide crystals. The attachments may have varying textures. The attachments may be structured to be permanent in nature or disposable in nature. Permanent attachments may further comprise the shaft being made of metal, hard plastic, such as polypropylene, or other suitable material and being integral with the attachment. Permanent attachments may further comprise larger quantities of abrasive material than the disposable attachments. Indeed, the disposable attachment may comprise relatively little abrasive material, or just enough to treat the skin of one customer or person. Then, after treatment, the disposable attachment may be discarded. Disposable attachments are also structured to attach to the shaft that can be integral to the body portion of the device, such that only the attachment need be disposed of thus further reducing cost.

Another aspect of the present invention may further comprise housing portions in the body portion of the device to allow the attachments to be repeatedly and releasably stored on the body portion while the attachment is not in use. The housing portions thus provide a temporary storage for those attachments that the user chooses not to use at any particular time. Then, when the user decides to switch out the attachment that is currently in use for an attachment housed in the housing portion, the user may make the switch without having to conduct a search for additional attachments.

The foregoing and other features and advantages of the present invention will be apparent from the following more detailed description of the particular embodiments of the invention, as illustrated in the accompanying drawings.

### BRIEF DESCRIPTION OF THE DRAWINGS

FIG. 1 is a side view of a hand-held skin-care device in accordance with the present invention.

FIG. 2 is a side view of components of the hand-held skin-care device in accordance with the present invention.

FIG. 3 is a side view of the hand-held skin-care device in accordance with an embodiment of the present invention.

FIG. 4 is a side view of components of the hand-held skin-care device in accordance with an embodiment of the present invention.

FIG. 5 is a top view of a component of the hand-held skin-care device in accordance with an embodiment of the present invention.

FIG. 6 is a perspective view of a hand-held skin-care device in accordance with an embodiment of the present invention.

### DETAILED DESCRIPTION OF EMBODIMENTS OF THE INVENTION

As discussed above, embodiments of the present invention relate to grooming products. More particularly, the present invention relates to a device and method for removing calluses and dead skin from areas of the body.

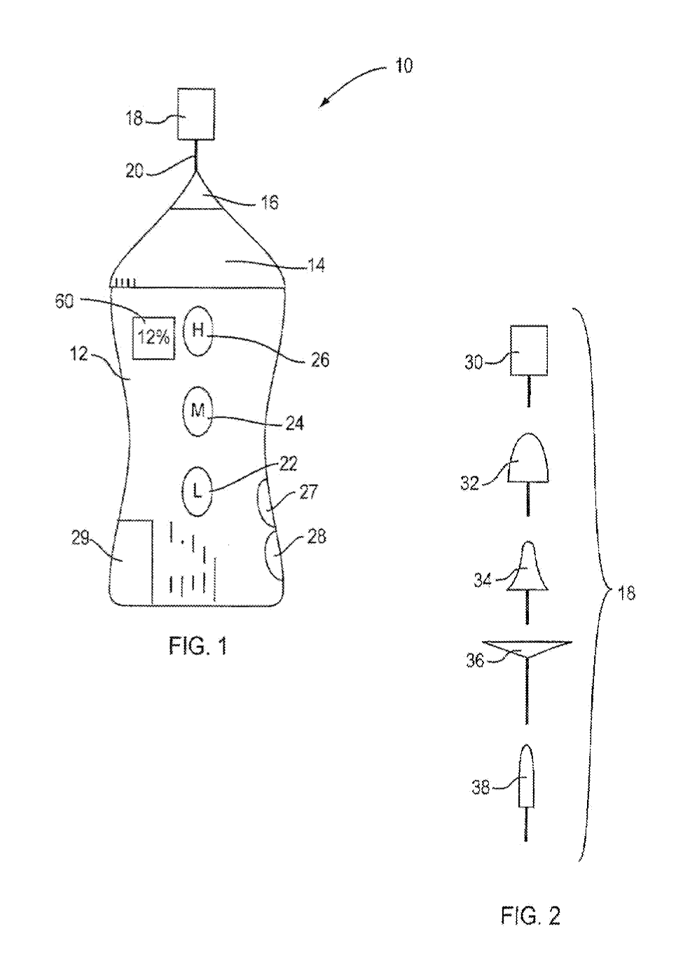

As shown in FIG. 1, the hand-held skin-care device 10 includes a body portion 12, a neck portion 14, and an attachment portion 16. The skin-care device 10 further includes an on/off control 28 and speed controls 22, 24 and 26 positioned on the body portion 12. The attachment portion 16 is structured to receive a variety of attachments 18, shown in FIG. 2. The attachments 18 are capable of repeatedly and releasably coupling to the skin-care device 10 at the attachment portion 16 via a shaft 20. The shaft 20 is structured to repeatedly and releasably couple to a motor (not pictured), the motor being housed entirely within the body portion 12. The motor serves to provide rotary motion to any attachment 18 coupled to the shaft 20. That is, under the condition that the motor is activated, the motor rotates the shaft 20 about its longitudinal axis which thus rotates the attachment 18 connected thereto about its own longitudinal axis. The rotational motion of the attachment 18 allows the user to quickly and conveniently remove dry skin by applying the spinning attachment 18 to the surface of the skin to buff away any unwanted dry skin and/or calluses. Also, the motor may also provide a vibration motion in addition to the rotary motion. The user may activate the vibration motion by pressing a vibration control 27 on the body portion 12.

As further shown in FIG. 1, the body portion 12 can be cylindrical in shape and have concave, or contoured, portions in the sides thereof to create an ergonomic feel in a user's hand. The body portion 12 further comprises on/off control 28 and speed controls 22, 24 and 26. The on/off control 28 serves to actuate the device 10. That is, when the on/off control 28 is pressed, power is supplied to the motor to actuate the motor. Under the condition that power is supplied to the motor, a portion, or all, of the on/off control 28 may light up to indicate to the user that device 10 is powered on. After use, the device 10 may be powered down by again pressing the on/off control 28. Speed controls 22, 24 and 26 control the rotational speed of the motor and thus the attachments 18. Speed control 22 is a low-speed control, speed control 24 is a medium-speed control, and speed control 26 is a high-speed control. Speed controls 22, 24, and 26 allow the user to set the rotational speed of the attachments 18 to one of three desired operational speeds by pressing the respective speed controls, 22, 24, and 26.

The skin-care device 10 can be powered through a power cord (not pictured) that can be repeatedly and releasably connected from the skin-care device 10 to a conventional standard 110V, 60 Hz power outlet. The skin-care device 10 can also be powered by a battery (not pictured) housed within a battery chamber 29 in the body portion 12. The battery may be rechargeable or non-rechargeable. If a rechargeable battery is used, the rechargeable battery may be charged by connecting the power cord from the skin-care device 10 to the standard power outlet. The rechargeable battery continues to charge whether the skin-care device 10 is concurrently in use or not, until the rechargeable battery reaches its full charge. Alternatively, the rechargeable battery can be charged by placing the skin-care device 10 on a base (not pictured) that is separate and apart from the skin-care device 10. The base serves as a charging station on which the skin-care device 10 can be placed to recharge the rechargeable battery. In this case, the power cord is connected from the base to a standard power outlet, such that the rechargeable battery recharges when the skin-care device 10 is coupled to the base. The base has a charge light that indicates when the device 10 is connected to the base and receiving a charge. Moreover, the base also includes attachment holding portions that hold the various attachments 18 that can be used with the device 10 while the attachments 18 are not in use.

FIG. 1 further shows that the body portion 12 further includes a digital display 60. Digital display 60 can be an LED display and serves to display to the user the operational status of the device 10. The digital display 60 remains inactive until the device 10 is powered on, at which time the display 60 illuminates. However, the device 10 may also temporarily illuminate under the condition that the user operates any of the speed controls 22, 24, and 26 on the device 10, whether or not the device 10 is currently powered on. The digital display 60 is capable of concurrently displaying any of, for example, the rotational speed of the attachment 18, the percentage of charge remaining in the battery, or the time left to operate the device 10 until the charge in the battery is depleted. The digital display 60 can illuminate in various colors, one color indicating the device 10 is powered on, a second color indicating the charge in the battery is low, a third color to indicate that the charge in the battery is critically low, and a fourth color to indicate that the charge in the battery is full. Further still, the digital display 60 can also illuminate in a fifth color to indicate to the user that the battery in the device 10 is charging. Moreover, the display 60 can repeatedly flash any of the above colors to grab the user's attention. For example, the digital display 60 may temporarily flash when a color is initially introduced or when a color is about to change to another color.

As shown in FIG. 2, attachments 18 can be of various shapes and sizes. For example, attachments 18 can have a cylindrical-shaped head 30, a rounded large-nose convex head 32, a rounded small-nose concave head 34, a disk-shaped head 36, and a rounded cylindrical-shaped thin head 38. The heads, 30, 32, 34, 36 and 38 provide the user with a selection of shapes from which to choose that allow the user to reach the hard-to-reach places that might not be accessible with a universally-shaped head. The heads, 30, 32, 34, 36 and 38 can be made of any material suitable for removing or buffing skin, for example, pumice (either natural or synthetic) or other abrasive material, such as aluminum oxide crystals. Moreover, the heads, 30, 32, 34, 36 and 38 can have varying textures along the axial length of the head, 30, 32, 34, 36 and 38. For example, the heads 30, 32, 34, 36 and 38 may have relatively soft abrasive material at the base of the head 30, 32, 34, 36 and 38 and rough, or course, abrasive material at the tip of the head 30, 32, 34, 36 and 38. Thus, in this configuration, as the head 30, 32, 34, 36 and 38 rotates about the shaft 20 the user has the option of applying the smooth section or the rough section, as desired.

A user can replace an attachment 18 currently coupled to the device 10 by releasing the shaft 20 from the attachment portion 16 and removing the shaft 20, and the accompanying attachment 18, from the device 10. The user may then attach another attachment 18 of her choice by coupling the shaft 20 of the respective attachment 18 to the attachment portion 16.

Attachments 18 can also be adapted for extended use or disposable use. When adapted for extended use, as shown in FIG. 2, each of the various heads, 30, 32, 34, 36 and 38 are fixedly coupled to their own respective shaft 20. The shaft 20 can be manufactured of rigid material, such as solid metal or plastic, to withstand repeated use. Moreover, the quantity of material comprising the heads 30, 32, 34, 36 and 38 in the extended-use attachment 18 is significantly larger than the quantity of material comprising a disposable head 40, as shown in FIG. 4. A larger quantity of material around the shaft 20 allows the extended-use heads 30, 32, 34, 36, and 38 to be repeatedly used before being worn down to the point that they need to be discarded.

On the other hand, as shown in FIG. 4, the attachment 18 can also be disposable in nature. Specifically, disposable head 40 is separate and apart from the shaft 20, but is structured to releasably and repeatedly couple to the shaft 20. Disposable head 40 is merely an example of a disposable head and any of the various shapes and sizes of the heads 30, 32, 34, 36 and 38 described above can be used as a disposable head. Structuring the disposable head 40 of FIG. 4 to be separate and apart from the shaft 20 allows manufacturers to manufacture a single shaft 20 to which any of the disposable heads 40 can be attached. Manufacturing a single shaft 20 instead of manufacturing a shaft 20 for each of the various shapes of the disposable heads reduces the manufacturing costs of the disposable attachments 18. To further reduce costs, the shaft 20 to be attached to the disposable head 40 can be hollowed out to reduce the amount of material required for suitable use.

Moreover, as a further cost reduction, the disposable head 40 can be manufactured with relatively small quantities of abrasive material, as compared with the amount of abrasive material used in attachments 18 of the extended-use variety. For example, to minimize the amount of material used in the disposable head 40, but yet to retain the overall shape of the disposable head 40 to mimic the overall shape of the extended-use heads 30, 32, 34, 36 and 38, the disposable head 40 may be manufactured to have a thin lining of material 44 placed over a larger plastic inner core 42, as shown in FIG. 5. In other words, the disposable head 40 may comprise a predetermined inner-shaped core 42 that mimics the overall shape of the extended-use heads 30, 32, 34, 36 and 38 and has only a thin layer of material 44 covering the exterior of the inner-shaped core 42. Thus, only a relatively small amount of material 44 is required to maintain the outer diameter shape of the disposable head 40 to be equivalent to the outer diameter shapes of the counterpart extended-use heads 30, 32, 34, 36 and 38. A smaller amount of material 44 on the disposable head 40 allows the manufacturer to make a cheaper attachment 18 and provides the user with greater convenience. For example, an attachment 18 having a disposable, single-use head 40 that functions equivalently to the extended-use attachments 18 offers the user a benefit of being able to use the disposable attachment 18 once and thereafter conveniently discard the attachment 18 without worrying that excessive, unused material 44 is left on the head 40. Such convenience is necessary in professional facilities, such as salons, health-care facilities, and health spas, where skin care products are only used on a one-time basis on an individual customer to maintain sanitary standards.

In certain embodiments, the device 10 may be manufactured with the shaft 20 being fixedly coupled to the attachment portion 16. In such an embodiment, only attachments 18 having disposable head 40 may be used.

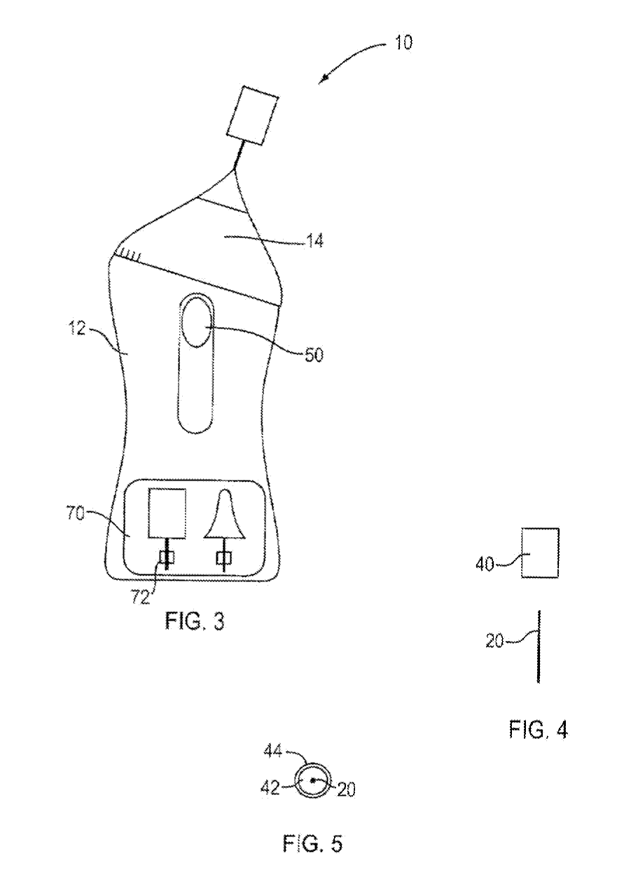

As shown in FIG. 3, the body portion 12 also includes housing portion 70 that is structured to temporarily store a plurality of attachments 18. Housing portion 70 is an indention in the side of the body portion 12 that is structured to receive the various shapes of the attachments 18. Alternatively, housing portion 70 can include a plurality of separate housing portions 70, or indentions, each of which can be structured to receive a single specific shape of an attachment 18. The housing portion 70 includes notches 72 attached thereto into which the respective shafts 20 of the attachments 18 may be pressed to releasably couple the attachments 18 to the notches 72. Under the condition that the attachments 18 are releasably coupled to the notches 72 within the housing portion 70, the attachments 18 are secured to the device 10 and do not protrude beyond the plane of the external radial surface of the body portion 12. Thus, the attachments 18 within the housing portion 70 will not catch or snag on objects, skin, or clothing as the device 10 is being used, stored, or transported.

As also shown in FIG. 3, certain embodiments of the present invention contemplate that the device 10 may be operated by a combined control 50 that both powers on and off the device as well as adjusts the rotational speed of the device. Further combined control 50 may also activate the vibration control of the device 10. Combined control 50 slides back and forth (or up and down) along the surface of the body portion 12. The sliding movement of the combined control 50 allows the user to dynamically adjust the rotational speed of the motor and thus the attachment 18. Indeed, the combined control 50 allows the user to dynamically increase or decrease the rotational speed of the motor between an upper-end rotational speed and a lower-end rotational speed as the combined control is respectively moved from one extreme end of its movement to the other extreme end of its movement. Accordingly, the user may fine tune the rotational speed of the attachment 18 to the user's preference. Every attachment 18 will likely have an optimal rotational speed that varies from user to user based upon the texture of the user's skin. Therefore, the combined control 50 allows each user to dynamically and delicately adjust the rotational speed of each attachment 18, as desired, during use of the device 10.

As shown in FIG. 3, the neck portion 14 is structured to incrementally pivot on the body portion 12 to allow the attachment 18 to be angled at various angles with respect to the body portion 12. Angling the attachment 18 allows the user to reach those places on her body that she would not otherwise be able to reach, or might be uncomfortable to reach if the neck portion 14 were not designed to pivot. Under the condition that the neck portion 14 is pivoted at an angle with respect to the body portion 12, a gear mechanism (not pictured), internal to the body portion 12, allows the rotational and vibration movement of the motor to transfer to the shaft 20. Thus, the attachment 18 rotates and vibrates the same whether or not the neck portion 14 is angled with respect to the body portion 12.

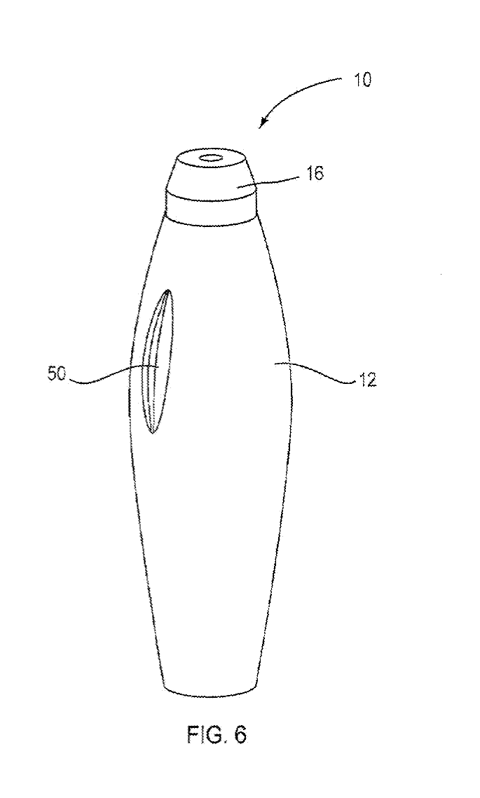

As shown in FIG. 6, certain embodiments of the device 10 comprise the body portion 12 being coupled directly to the attachment portion 16 without the neck portion 14 placed therebetween. Moreover, the body portion 12 may have a cylindrical shape with convex, contoured sides; the device 10 measures approximately 6 inches in length and 2 inches in diameter.

As shown in FIGS. 1, 3 and 6, the skin-care device 10 can be waterproof, such that the user can operate the device 10 in wet environments, such as, for example, the shower or the bath. The battery chamber 29, the digital display 60, and the combined control 50 on the body portion 12 are each also structured to be waterproof, such that the buttons operate under wet conditions and do not allow wetness or moisture to enter within the body portion 12. The housing portion 70 on the body portion 12 is also waterproof, such that water cannot penetrate into the body portion 12 through the housing portion 70. Moreover, the intersection of the shaft 20 and the attachment portion 16 is also waterproof, such that the user can interchange attachments 18 in wet conditions without wetness or moisture entering the device 10. With such features, the user can be confident that the device 10 will function properly when operating the device 10 in wet environments. In addition, the surface of the body portion 12 may also be textured to provide for better grip during wet conditions. The texture may be indentions in the surface of the body portion 12. The indentions may be patterns, such as grooves set at predetermined intervals, concentric circles, or the like. Alternatively, the texture may also be raised portions, such as bumps or ridges. The body portion 12 may also include a rubber grip for the user's hand. The grip may be smooth, or may include texture similar to the texture of the body portion 12. In certain embodiments, the rubber grip is contoured or structured in a shape that conveniently receives a user's hand.

The device 10 is also structured to receive a cap that can be placed over the attachments 18 to protect the attachments 18 when the device 10 is not in use or is being transported by the user, for example, in the user's bag. The cap is structured to be placed over and completely cover the attachments 18, and the cap releasably couples to the device 10 on one of the body portion 12, the neck portion 14, or the attachment portion 16.

The method of treating skin by using the skin-care device 10 is simple and straight forward. The user selects the appropriate attachment 18 and powers on the device 10. The user then selects the appropriate operational speed of the device 10 to perform the desired task. For example, on soft skin a stow speed may be more useful, whereas on hard or callused skin it may be necessary to operate the device 10 at high speed to achieve the desired result. The user may also apply a lotion, cream, or solution to the head 30, 32, 34, 36 and 38 of the attachment 18, the skin to be treated, or both, prior to powering on the device 10 to assist in the treatment of the skin. Lotions, creams, and solutions can act as a lubricant between the attachment 18 and the user's skin to more efficiently treat the skin.

After powering on the device and selecting the appropriate speed, the attachment 18 may be pressed against the skin, as desired by the user, to allow the rotary motion and/or vibration motion of the attachment 18 to work its magic. For example, if the user wishes to smooth the thick, cracked skin on the heels of her feet, she could operate the device 10 on the high speed, thereby quickly sloughing away layers of dead skin. Similarly, the user who wishes to smooth the fragile, dry skin of her elbows or hands could operate the device 10 at the low or medium speed, thus efficiently removing dry, flaky skin cells in a safe and gentle manner. The user can perform both of the above tasks without having to power down the device, because the device 10 is capable of changing speeds during operation by operating the speed controls 22, 24, and 26 or the combined control 50. Furthermore, if desired, the user can treat one area of skin, power off the device 10, change the attachment 18, power up the device 10 and continue treatment of the skin with the newly added attachment 18. If desired, the user may also perform the above steps while showering or taking a bath. Moreover, the user may perform the above steps immediately after soaking in water the areas of the skin to be treated by the device 10.

After treating the skin with the device 10, the user can rinse the device 10 and the attachments 18 with water to clean the device 10 prior to recharging or storing the device 10. The user may also choose to apply the lotion, cream, or solution to the areas of the skin that have been treated by the device 10 to further enhance the treatment.

The embodiments and examples set forth herein were presented in order to best explain the present invention and its practical application and to thereby enable those of ordinary skill in the art to make and use the invention. However, those of ordinary skill in the art will recognize that the foregoing description and examples have been presented for the purposes of illustration and example only. The description as set forth is not intended to be exhaustive or to limit the invention to the precise form disclosed. Many modifications and variations are possible in light of the teachings above without departing from the spirit and scope of the present invention.

1. A waterproof hand-held skin care device, comprising:
a body portion with a plurality of sides;
a neck portion disposed above said body portion;
an attachment portion with a vertical shaft;
a plurality of speed control buttons positioned on said body portion;
an on and off control button positioned on said body portion;
a variety of attachments with a head with a base and a tip that are repeatedly and releasably coupled to said attachment portion via said vertical shaft that rotates said attachments;
a battery chamber in said body portion that houses a battery that powers said tool; and
a digital display that serves to display an operational status of said device.

2. The device according to claim 1, wherein said body portion is cylindrical in shape and has a plurality of concave or contoured portions on said sides to create an ergonomic feel in a user's hand.

3. The device according to claim 1, wherein said body portion includes a housing portion that is structured to temporarily store said attachments.

4. The device according to claim 3, wherein said housing portion includes a plurality of notches where said attachments are pressed into said notches to releasably couple said attachments to said notches.

5. The device according to claim 1, wherein said neck portion is structured to incrementally pivot on said body portion to allow said attachment to be angled at a plurality of various angles with respect to said body portion.

6. The device according to claim 1, wherein sad shaft is made of material selected from the group consisting of metal, hard plastic or polypropylene.

7. The device according to claim 1, wherein said speed control buttons include a low speed control, a medium speed control and a high speed control.

8. The device according to claim 1, wherein said attachments are selected from the croup consisting of a cylindrical-shaped head, a rounded large-nose convex head, a rounded small-nose concave head, a disk-shaped head or a rounded cylindrical-shaped thin head.

9. The device according to claim 1, wherein said attachments are vibrated by said shaft.

10. The device according to claim 9, wherein said attachments are vibrated and rotated by said shaft.

11. The device according to claim 1, wherein said head is made of material selected from the group consisting of natural pumice, synthetic pumice, pumice or aluminum oxide crystals.

12. The device according to claim 1, wherein said attachments have soft abrasive material at said head and rough or abrasive material at said tip.

13. The device according to claim 1, wherein said attachments and said head are disposable.

14. The device according to claim 13, wherein said disposable head is manufactured to have a thin lining of said material disposed over a larger plastic core to make a cheaper said attachment and provide greater convenience.

15. The device according to claim 1, wherein said shaft is hollowed out to reduce material.

16. The device according to claim 1, wherein sad battery is a rechargeable battery.

17. The device according to claim 16, wherein said rechargeable battery continues to charge when said device is concurrently in use until said rechargeable battery reaches its full charge.

18. The device according to claim 1, wherein said device is powered by and connected to a conventional standard power outlet.

19. The device according to claim 1, wherein said digital display displays a rotational speed of said attachment, a percentage of charge remaining in said battery and time left to operate said device until said charge in said battery is depleted.

20. The device according to claim 1, wherein said device removes calluses and dead skin from a plurality of areas of said user's body.
